(19) United States
(12) Patent Application Publication
(10) Pub. No.: US 2019/0016652 A1
(43) Pub. Date: Jan. 17, 2019

(54) SYSTEMS AND METHODS FOR MANUFACTURING AND SEPARATING (Z)-1-CHLORO-3,3,3-TRIFLUOROPROPENE

(71) Applicant: Honeywell International Inc., Morris Plains, NJ (US)

(72) Inventors: Daniel C. Merkel, Orchard Park, NY (US); Peter Scheidle, Wheatfield, NY (US); Haiyou Wang, Amherst, NY (US); Christian Jungong, Depew, NY (US)

(21) Appl. No.: 16/026,149

(22) Filed: Jul. 3, 2018

Related U.S. Application Data
(60) Provisional application No. 62/531,699, filed on Jul. 12, 2017.

(51) Int. Cl. C07C 17/358

(52) U.S. Cl. CPC C07C 17/358; C07B 2200/09

(57) ABSTRACT

The present disclosure includes various manufacturing and separation processes for the production of (Z)-1-chloro-3,3,3-trifluoropropene from (E)-1-chloro-3,3,3-trifluoropropene. The efficient separation of (Z)-1-chloro-3,3,3-trifluoropropene from unreacted (E)-1-chloro-3,3,3-trifluoropropene may allow for the ability to recycle unreacted starting materials and to maximize raw material utilization and product yields.

### CROSS REFERENCE TO RELATED APPLICATIONS

This application claims the benefit under Title 35, U.S.C. § 119(e) of U.S. Provisional Patent Application Ser. No. 62/531,669, entitled SYSTEMS AND METHODS FOR MANUFACTURING AND SEPARATING (Z)-1-CHLORO-3,3,3-TRIFLUOROPROPENE, filed on Jul. 12, 2017, the entire disclosure of which is expressly incorporated by reference herein.

### FIELD OF THE DISCLOSURE

This disclosure relates to the manufacture of (Z)-1-chloro-3,3,3-trifluoropropene. More specifically, this disclosure relates to manufacturing processes and separation processes of (Z)-1-chloro-3,3,3-trifluoropropene from (E)-1-chloro-3,3,3-trifluoropropene.

### BACKGROUND

Fluorocarbon based fluids have found widespread use in industry in a number of applications, including use as refrigerants, aerosol propellants, blowing agents, heat transfer media, and gaseous dielectrics. Due to suspected environmental problems associated with the use of some of these fluids, including the relatively high global warming potentials associated therewith, it is desirable to use fluids having the lowest possible global warming potential (GWP) in addition to also having zero ozone depletion potential (ODP). Thus, there is considerable interest in developing environmentally friendlier materials for the applications mentioned above.

Hydrochlorofluoroolefins (HCFOs) having zero ozone depletion and low global warming potential have been identified as potentially filling this need. However, the toxicity, boiling point, and other physical properties of such chemicals vary greatly from isomer to isomer. One HCFO having valuable properties is 1-chloro-3,3,3-trifluoropropene (HCFO-1233zd or simply 1233zd), which has been proposed as a next generation non-ozone depleting and low global warming potential solvent.

1233zd has two isomers: (E) and (Z). HCFO-1233zd(E) or (E)-1-chloro-3,3,3-trifluoropropene has a boiling point of approximately 19° C. while HCFO-1233zd(Z) or (Z)-1-chloro-3,3,3-trifluoropropene has a boiling point of approximately 39° C. While 1233zd(E) is a HCFO having valuable properties, in some instances, a zero ozone depletion and low global warming potential HCFO with a higher boiling point may be desired. Thus, 1233zd(Z) may be a suitable HCFO when higher boiling points are desired.

1233zd(E) is a known reactant in the formation of 1233zd(Z). However, these processes are often carried out under very high pressures and/or temperatures, usually in excess of 400° C. High pressures and temperatures can, in some instances, create lower yields in the manufacture of (Z)-1-chloro-3,3,3-trifluoropropene and can also lead to side products, such as HF or HCl.

Thus, a need exists for the ability to more efficiently manufacture (Z)-1-chloro-3,3,3-trifluoropropene from (E)-1-chloro-3,3,3-trifluoropropene. Also, additional purification methods of more efficiently separating (Z)-1-chloro-3,3,3-trifluoropropene from (E)-1-chloro-3,3,3-trifluoropropene and side products are needed.

### SUMMARY

The present disclosure includes various manufacturing and separation processes for the production of (Z)-1-chloro-3,3,3-trifluoropropene from (E)-1-chloro-3,3,3-trifluoropropene. Due to the increased relative thermodynamic stability of (E)-1-chloro-3,3,3-trifluoropropene to (Z)-1-chloro-3,3,3-trifluoropropene, the manufacture of (Z)-1-chloro-3,3,3-trifluoropropene is typically accomplished by using (E)-1-chloro-3,3,3-trifluoropropene as a reactant. In some applications, (Z)-1-chloro-3,3,3-trifluoropropene is often desired and used in industry due to various properties, such as a higher boiling point.

Also, (E)-1-chloro-3,3,3-trifluoropropene has a boiling point of approximately 19° C. while (Z)-1-chloro-3,3,3-trifluoropropene has a boiling point of approximately 38° C. This surprising difference in boiling points between these two isomers and their difference in their relative thermodynamic stability may allow for efficient separation of (Z)-1-chloro-3,3,3-trifluoropropene from unreacted (E)-1-chloro-3,3,3-trifluoropropene when both isomers are manufactured from the reactant (E)-1-chloro-3,3,3-trifluoropropene. The efficient separation of (Z)-1-chloro-3,3,3-trifluoropropene from unreacted (E)-1-chloro-3,3,3-trifluoropropene may allow for the ability to recycle unreacted starting materials and to maximize raw material utilization and significantly improve product yields.

For example, in such separation processes, after production in an isomerization reactor, (Z)-1-chloro-3,3,3-trifluoropropene may be separated from (E)-1-chloro-3,3,3-trifluoropropene through one or more purification processes, such as distillation and/or scrubbing.

Exemplary methods may of manufacturing (Z)-1-chloro-3,3,3-trifluoropropene may include contacting a reactant fluid stream containing (E)-1-chloro-3,3,3-trifluoropropene with a catalyst to form a reacted fluid stream comprising (Z)-1-chloro-3,3,3-trifluoropropene, contacting the reacted fluid stream with a scrubbing fluid to remove a byproduct from the reacted fluid stream, removing the scrubbing fluid from the reacted fluid stream, separating (Z)-1-chloro-3,3,3-trifluoropropene from unreacted (E)-1-chloro-3,3,3-trifluoropropene in the reacted fluid stream, and recycling the unreacted (E)-1-chloro-3,3,3-trifluoropropene with the reactant fluid stream containing (E)-1-chloro-3,3,3-trifluoropropene. In some embodiments, the separating (Z)-1-chloro-3,3,3-trifluoropropene from unreacted (E)-1-chloro-3,3,3-trifluoropropene in the reacted fluid stream is done by distillation. For example, the distillation may be done with a plurality of distillation columns, for example the distillation columns may be in series or in parallel.

In various embodiments, the scrubbing fluid may include water, a low-caustic composition, or a mixture thereof. To remove the scrubbing fluid, the scrubbed fluid may be dried, for example with a dryer. In some embodiments, the dryer comprises a desiccant. In some embodiments, the dryer may include molecular sieves, a mesoporous material, a macroporous material, inorganic anhydrites, or a combination thereof.

The catalysts of the enclosed embodiments are not particularly limited and may be a high-temperature catalyst or a low temperature catalyst. Exemplary low temperature catalysts include chromium based low temperature catalysts, an aluminum based low temperature catalysts, halogenated metals, metal oxides, or combinations thereof. Exemplary chromium based low temperature catalyst and the chromium based low temperature catalysts is chromium oxide, chromium oxyfluoride, chromium fluoride, or a combination thereof. Thus, in various embodiments, the contacting the fluid stream containing (E)-1-chloro-3,3,3-trifluoropropene with the catalyst to form the reacted fluid stream comprising (Z)-1-chloro-3,3,3-trifluoropropene may be done at a temperature below about 600° C. and may be done at an operating pressure between about 4 psia to about 214 psia.

Other methods of manufacturing (Z)-1-chloro-3,3,3-trifluoropropene may include contacting a reactant stream containing (E)-1-chloro-3,3,3-trifluoropropene with a catalyst to form a reacted fluid stream containing (Z)-1-chloro-3,3,3-trifluoropropene, separating (Z)-1-chloro-3,3,3-trifluoropropene from unreacted (E)-1-chloro-3,3,3-trifluoropropene in the reacted fluid stream to form a separated reacted fluid stream containing unreacted (E)-1-chloro-3,3,3-trifluoropropene, contacting the separated reacted fluid stream containing unreacted (E)-1-chloro-3,3,3-trifluoropropene with a scrubbing fluid to remove one or more byproducts from the separated reacted fluid stream, removing the scrubbing fluid from the separated reacted fluid stream containing unreacted (E)-1-chloro-3,3,3-trifluoropropene, and recycling the unreacted (E)-1-chloro-3,3,3-trifluoropropene with the reactant fluid stream containing (E)-1-chloro-3,3,3-trifluoropropene. Some embodiments may also include separating hydrogen halides from the separated reacted fluid stream containing unreacted (E)-1-chloro-3,3,3-trifluoropropene. For example, in various embodiments, the hydrogen halides may be separated from the separated reacted fluid stream containing unreacted (E)-1-chloro-3,3,3-trifluoropropene after the separated reacted fluid stream containing unreacted (E)-1-chloro-3,3,3-trifluoropropene is contacted with the scrubbing fluid.

In some embodiments, the separating (Z)-1-chloro-3,3,3-trifluoropropene from unreacted (E)-1-chloro-3,3,3-trifluoropropene in the reacted fluid stream may be performed by distillation, for example, with a plurality of distillation columns (e.g., a plurality of distillation columns in series).

### BRIEF DESCRIPTION OF THE DRAWINGS

The above mentioned and other features and objects of this disclosure, and the manner of attaining them, will become more apparent and the disclosure itself will be better understood by reference to the following description of exemplary embodiments of the disclosure taken in conjunction with the accompanying drawings, wherein:

FIG. 1 is a process flow diagram showing an exemplary manufacturing process of (Z)-1-chloro-3,3,3-trifluoropropene having a recycle stream of unreacted (E)-1-chloro-3,3,3-trifluoropropene;

FIG. 2 is a process flow diagram showing an exemplary manufacturing process of (Z)-1-chloro-3,3,3-trifluoropropene using an additional distillation column to separate the overhead stream after scrubbing the isomerization products;

FIG. 3 is a process flow diagram showing an exemplary manufacturing process of (Z)-1-chloro-3,3,3-trifluoropropene using a plurality of distillation columns in series after scrubbing the isomerization products for both the overhead and bottoms;

FIG. 4 is a process flow diagram showing an exemplary manufacturing process of (Z)-1-chloro-3,3,3-trifluoropropene where with both a bottoms stream and a stripping stream;

FIG. 5 is a process flow diagram showing an exemplary manufacturing process of (Z)-1-chloro-3,3,3-trifluoropropene that includes at least a second column to separate the overhead of the first distillation column series before the separated product stream is scrubbed;

FIG. 6 is a process flow diagram showing an exemplary manufacturing process of (Z)-1-chloro-3,3,3-trifluoropropene includes a plurality of distillation columns to separate both the overhead and the bottoms of the first distillation column before the separated product stream is scrubbed;

FIG. 7 is a process flow diagram where the distillation columns are used to separate a portion of the product stream before and after scrubbing;

FIG. 8 is a process flow diagram showing an exemplary process similar to FIG. 7 where the bottoms of the first distillation column are further separated by a second distillation column;

FIG. 9 is a process flow diagram similar to FIG. 8, but where the scrubbed (E)-1-chloro-3,3,3-trifluoropropene is recycled directly after drying; and

FIG. 10 is a process flow diagram where the first distillation column contains a bottoms stream and a stripping section stream.

Corresponding reference characters indicate corresponding parts throughout. Although the drawings represent various embodiments of the present disclosure, the drawings are not necessarily to scale and certain features may be exaggerated in order to better illustrate and explain various aspects the present disclosure. The exemplification set out herein illustrates exemplary embodiments of the disclosure, in various forms, and such exemplifications are not to be construed as limiting the scope of the disclosure in any manner.

### DETAILED DESCRIPTION

As briefly described above, this disclosure provides for the manufacture and separation of (Z)-1-chloro-3,3,3-trifluoropropene (HCFO-1233zd(Z) or simply 1233zd(Z)) from (E)-1-chloro-3,3,3-trifluoropropene (HCFO-1233zd(E) or simply 1233zd(E)) with the use of either a low temperature or high-temperature catalyst. Separation and recovery of (Z)-1-chloro-3,3,3-trifluoropropene, unreacted (E)-1-chloro-3,3,3-trifluoropropene, and other useful products of side reactions is desirable because it allows for the simultaneous utilization of (Z)-1-chloro-3,3,3-trifluoropropene, recycling of (E)-1-chloro-3,3,3-trifluoropropene, recycling products of side reactions (e.g., HF, HCl, etc.), and reutilization of catalysts that can be produced as side products of other manufacturing processes.

(Z)-1-chloro-3,3,3-trifluoropropene can be produced from reacting (E)-1-chloro-3,3,3-trifluoropropene in the presence of either a low temperature or a high-temperature catalyst. The (Z)-1-chloro-3,3,3-trifluoropropene can then be separated from unreacted (E)-1-chloro-3,3,3-trifluoropropene before or after contacting the reacted fluid stream with a scrubbing fluid.

The embodiments disclosed below are not intended to be exhaustive or limit the disclosure to the precise forms disclosed in the following detailed description. Rather, the embodiments are chosen and described so that others skilled in the art may utilize their teachings.

As used herein, the modifier “about” used in connection with a quantity is inclusive of the stated value and has the meaning dictated by the context (for example, it includes at least the degree of error associated with the measurement of the particular quantity). When used in the context of a range, the modifier “about” should also be considered as disclosing the range defined by the absolute values of the two endpoints. For example, the range “from about 2 to about 4” also discloses the range “from 2 to 4.”

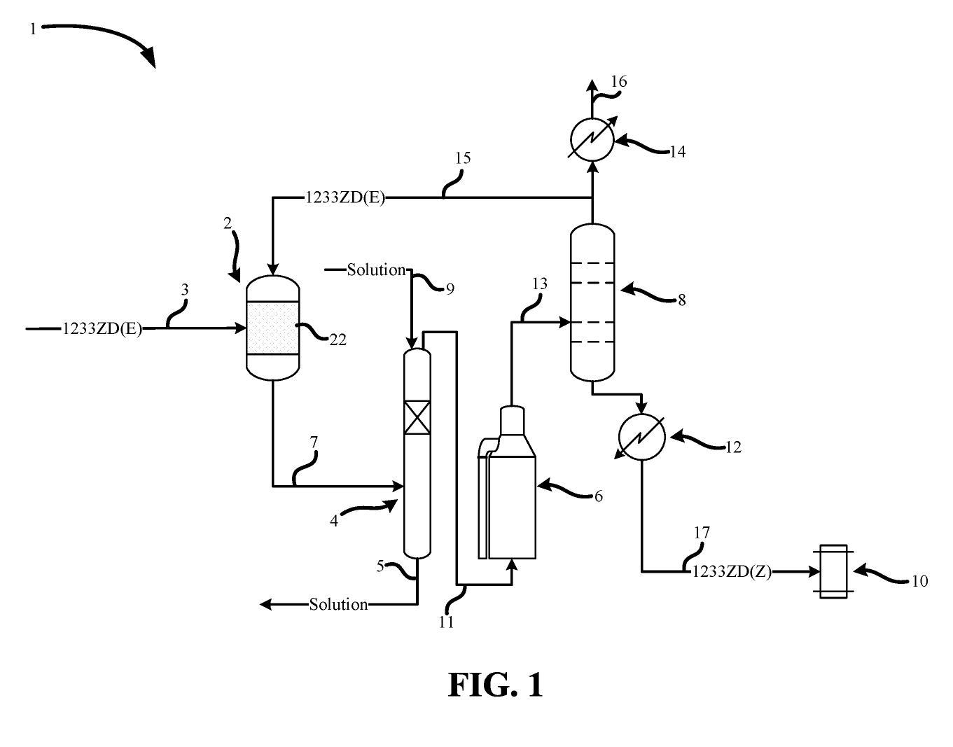

FIGS. 1-10 depict process flow diagrams embodying the present disclosure, and it should be understood that many features are common between the various diagrams. Although such common features are not repeated in the discussion below, any descriptions of features described in connection with one diagram or process that are common with another diagram or process are equally applicable to such other diagrams or processes.

FIG. 1 is a process flow diagram illustrating exemplary manufacturing process flow 1 according to various embodiments. Process flow 1 illustrates reactant stream 3 containing (E)-1-chloro-3,3,3-trifluoropropene (i.e., a fluid stream containing (E)-1-chloro-3,3,3-trifluoropropene). Reactant stream may contain various concentrations of (E)-1-chloro-3,3,3-trifluoropropene. Exemplary (E)-1-chloro-3,3,3-trifluoropropene ranges include greater than 90 wt. %, greater than 95 wt. %, and greater than 98 wt. %. The reactant stream 3 is then contacted with a catalyst 22 contained within isomerization reactor 2. Upon contact with the catalyst, the fluid stream containing (E)-1-chloro-3,3,3-trifluoropropene reacts with the catalyst to form a reacted fluid stream 7 comprising (Z)-1-chloro-3,3,3-trifluoropropene.

As used herein, the term “fluid” can include liquids, vapors, gasses, or a mixture of any combination of liquids, vapors, and/or gasses. Thus, for example, at least a portion of the fluid stream containing (Z)-1-chloro-3,3,3-trifluoropropene may contain (Z)-1-chloro-3,3,3-trifluoropropene vapors, (Z)-1-chloro-3,3,3-trifluoropropene gasses, and/or liquid (Z)-1-chloro-3,3,3-trifluoropropene.

The low volatile isomerization reactor product stream 7 may then be sent to a scrubber 4 where it is contacted with a scrubbing fluid, such as solution stream 9. In scrubber 4, side reactants having a lower boiling point than (E)-1-chloro-3,3,3-trifluoropropene (e.g., HCl and/or HF) that are reactive with scrubbing solution stream 9 may then be removed from the isomerization reactor via scrubber product stream 5, and may be used in other processes, recycled, or disposed of. The scrubbing solution is not particularly limited and may include water and/or weak caustic solutions, for example, the scrubbing solution may include deionized water, distilled water, or tap water or any suitable caustic solution with a pH below or equal to about 10, such as NaOH, KOH, or Ca(OH)₂.

After solution stream 9 is contacted with the low volatile isomerization reactor product stream 7 in scrubber 4, remaining scrubbing fluid contained in scrubbed/reacted fluid stream 11 may then be removed from scrubbed/reacted fluid stream 11, for example with dryer 6. Dryer 6 is not particularly limited and may include any known methods of drying, such as the use of molecular sieves (e.g., 3 A molecular sieves), microporous material (e.g., zeolites, porous glass, active carbon, clays, etc.), mesoporous material (e.g., silica gel), macroporous material (e.g., mesoporous silica), inorganic anhydrites (e.g., CaSO₄) and combinations thereof.

The dried, reacted, and scrubbed fluid stream 13 may then be undergo a separating process where (Z)-1-chloro-3,3,3-trifluoropropene is separated from unreacted (E)-1-chloro-3,3,3-trifluoropropene in the reacted fluid stream. For example, process flow 1 shows the separation of the dried, reacted, and scrubbed/dried fluid stream 13 using a distillation column 8. The overhead 1233zd(E) recycle stream 15, which may comprise a majority (greater than 50 wt. %) of (E)-1-chloro-3,3,3-trifluoropropene, may be recycled and reused by sending back to reactor 2, and purge valve 16 and reboiler 14 may be used to purge the system, while the bottoms stream 17, which may comprise a majority (greater than 50 wt. %) of (Z)-1-chloro-3,3,3-trifluoropropene, may be collected in 1233zd(Z) container 10.

The catalyst 22 contained within isomerization reactor 2 is not particularly limited and may be selected based upon various design parameters. As used herein, the term “catalyst” means a substance which affects the rate of a chemical reaction without itself being consumed or undergoing a chemical change. In some embodiments, the step of contacting the fluid stream containing (E)-1-chloro-3,3,3-trifluoropropene with the catalyst to form the reacted fluid stream comprising (Z)-1-chloro-3,3,3-trifluoropropene may be performed at a temperature between about 90° C. to about 600° C. Thus, as used herein, catalysts may include either “low temperature catalysts,” “high temperature catalysts”, or both.

Low temperature catalysts include catalysts that cause the isomerization of (E)-1-chloro-3,3,3-trifluoropropene to (Z)-1-chloro-3,3,3-trifluoropropene at a temperature below 300° C., such as between about 90° C. to about 300° C., between about 150° C. to about 300° C., between about 200° C. to about 275° C., between about 200° C. to about 250° C., or about 225° C. Exemplary low temperature catalysts include chromium-based low temperature catalysts, aluminum-based low temperature catalysts, halogenated metals, metal oxides, or combinations thereof. Exemplary chromium-based low temperature catalysts include chromium oxide, chromium oxyfluoride, chromium fluoride, or combinations thereof. Other exemplary low temperature catalysts may include AlCl₃, AlF₃, alumina (Al₂O₃), NiF₂, fluorinated crystalline Cr₂O₃, unflorinated amorphous Cr₂O₃, FeCl₃/Carbon, or mixtures thereof.

High temperature catalysts include catalysts that cause the isomerization of (E)-1-chloro-3,3,3-trifluoropropene to (Z)-1-chloro-3,3,3-trifluoropropene at a temperature below 600° C., such as between about 300° C. to about 600° C., between about 300° C. to about 500° C., between about 300° C. to 450° C., between about 300° C. to about 400° C., or about 350° C. Exemplary high-temperature catalysts may include zero valent metals and alloys. Exemplary alloys include stainless steel, nickel-chromium based super alloys, and nickel-copper alloys (e.g., 316 SS, Inconel 625, Monel 400, etc.).

Catalysts (both high temperature catalysts and low temperature catalysts) can be either amorphous or crystalline. In some embodiments, catalysts may be recharged or replaced according to various embodiments. Further details and description of suitable catalysts may be found, for example, in U.S. Pat. Nos. 8,217,208, 9,162,947, and 9,650,320, all of which are each individually incorporated by reference herein in their entirety.

The operating pressure of the isomerization reactor is not particularly limited and may be between about 4 psia to about 214 psia, between about 4 psia to about 200 psia, between about 10 psia to about 150 psia, between about 25 psia to about 100 psia, or between about 25 psia to about 45 psia, or about 30 psia.

FIGS. 1-10 contain various process flow diagrams having similar or common features between them. It shall be understood that when the common features shown or described herein are previously described, while their features will be understood to be applied to all process flows having the same feature, discussion of the common features will not be repeated.

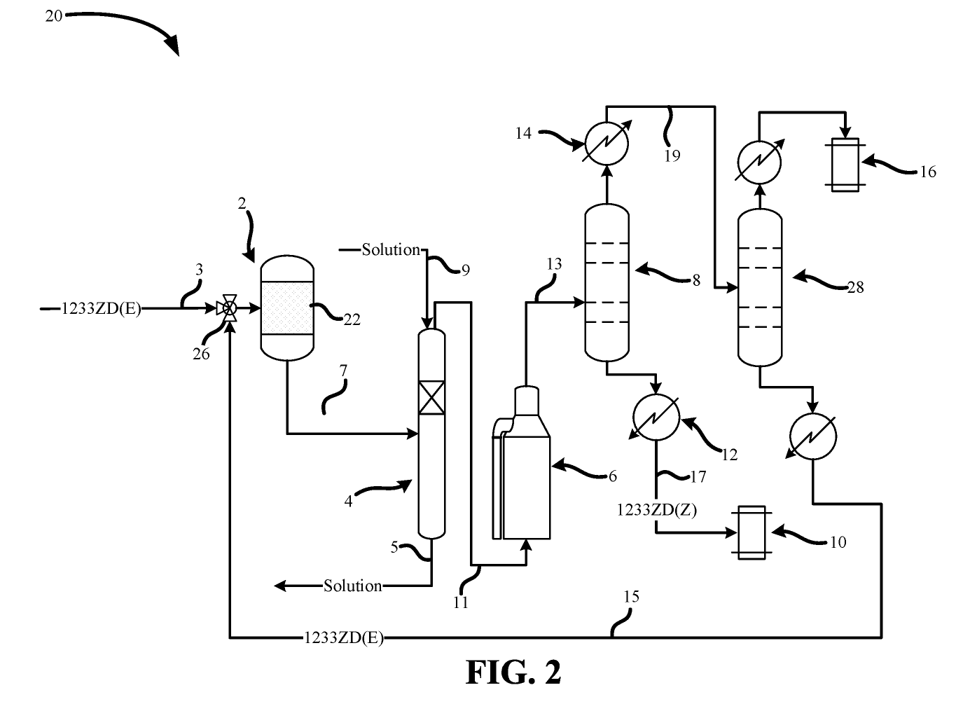

FIG. 2 is a process flow diagram showing an exemplary manufacturing process of (Z)-1-chloro-3,3,3-trifluoropropene using an additional distillation column to separate the overhead stream after scrubbing the isomerization product stream. Process flow 20 may have a similar overall process flow to that as shown in FIG. 1. In process flow 20 however, overhead stream 19 may pass either through a reboiler (e.g., reboiler 14) or cooler and then sent to distillation column 28 for further distillation. Bottoms stream 17 of distillation column 8 may be collected after passing through cooler 12, while the bottoms stream of distillation column 25 may pass through cooler or condenser 25 and then recycled back as 1233zd(E) recycle stream 15, which may contain separated (E)-1-chloro-3,3,3-trifluoropropene to be mixed with (E)-1-chloro-3,3,3-trifluoropropene reactant stream 3 via mixer valve 26 and re-sent to isomerization reactor 2 to improve reactant turnover and/or overall yield. Overhead stream 21 of distillation column may pass through reboiler or cooler 24 to then be collected in low boiling co-product collection container 18 and used in other processes, sold, or disposed of.

As used herein, the term “low boiling co-product” may be understood to include co-products of the isomerization of (Z)-1-chloro-3,3,3-trifluoropropene from (E)-1-chloro-3,3,3-trifluoropropene that have a boiling point of about 16° C. and below. For example, exemplary low boiling co-products may include HFC-245fa (1,1,1,3,3-pentafluoropropane having a boiling point of about 15° C., commercially available from HONEYWELL INTERNATIONAL INC., a New Jersey corporation), 1234ze (trans-1,3,3,3-tetrafluoropropene having a boiling point of about −19° C., commercially available from HONEYWELL INTERNATIONAL INC., a New Jersey corporation), 1234ze (cis-1,3,3,3-tetrafluoropropene having a boiling point of about 9° C.), and trifluoropropyne (having a boiling point of about −48° C.).

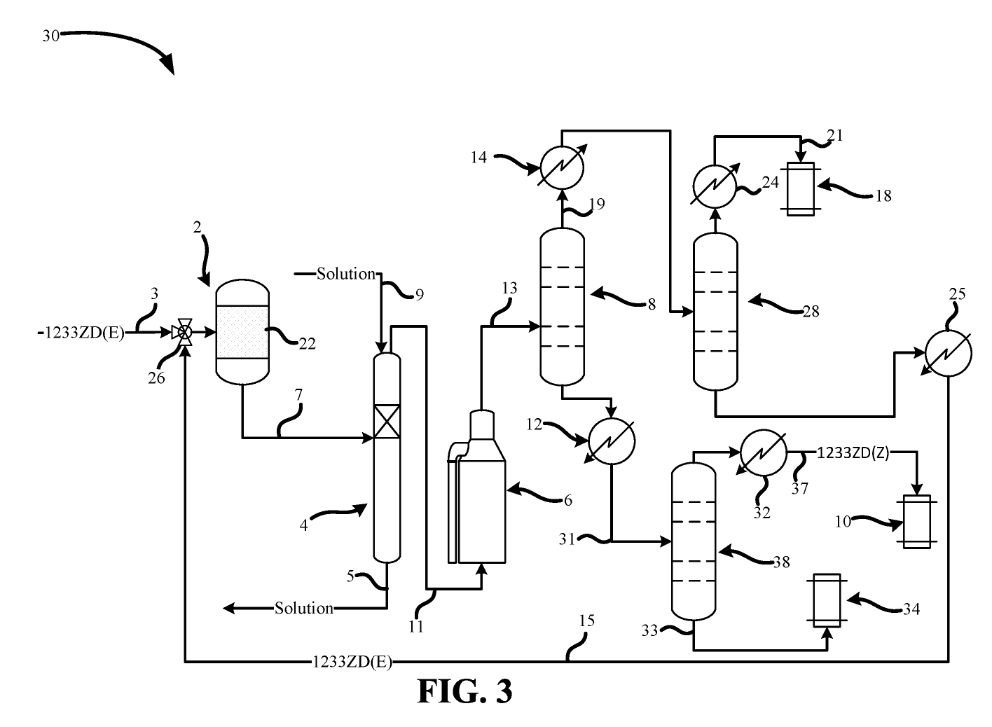

FIG. 3 is a process flow diagram showing an exemplary manufacturing process 30 of (Z)-1-chloro-3,3,3-trifluoropropene using a plurality of distillation columns in series after scrubbing the isomerization products for both the overhead and bottoms. Thus, the process flow diagram in FIG. 3 is similar to that shown in FIG. 2, but the process flow diagram depicted in FIG. 3 includes further distillation of the bottoms stream 31 of distillation column 8. Bottoms stream 31 may pass through cooler 12 and then sent to distillation column 38, where it may be further distilled to form overhead 1233zd(Z) stream 37, which may pass through cooler 32 and collected in 1233zd(Z) container 10, and bottoms high boiling co-products stream 33, which may be collected in high boiling co-products container 34.

As used herein, the term “high boiling co-products” may be understood to include co-products of the isomerization process comprising co-products having a boiling point of about 41° C. or higher. For example, some high boiling co-products may include HFC-244fa (3-chloro-1,1,1,3-tetrafluoropropane having a boiling point of about 42° C.), HFC-243fa (3,3-dichloro-1,1,1-trifluoropropane having a boiling point of about 73° C.), HCFO1232 isomers (dichlorodifluoropropene), and dimers of 1233zd (eg. 3-chloro-1,1,1,6,6,6-hexaflourohexa-2,4-diene).

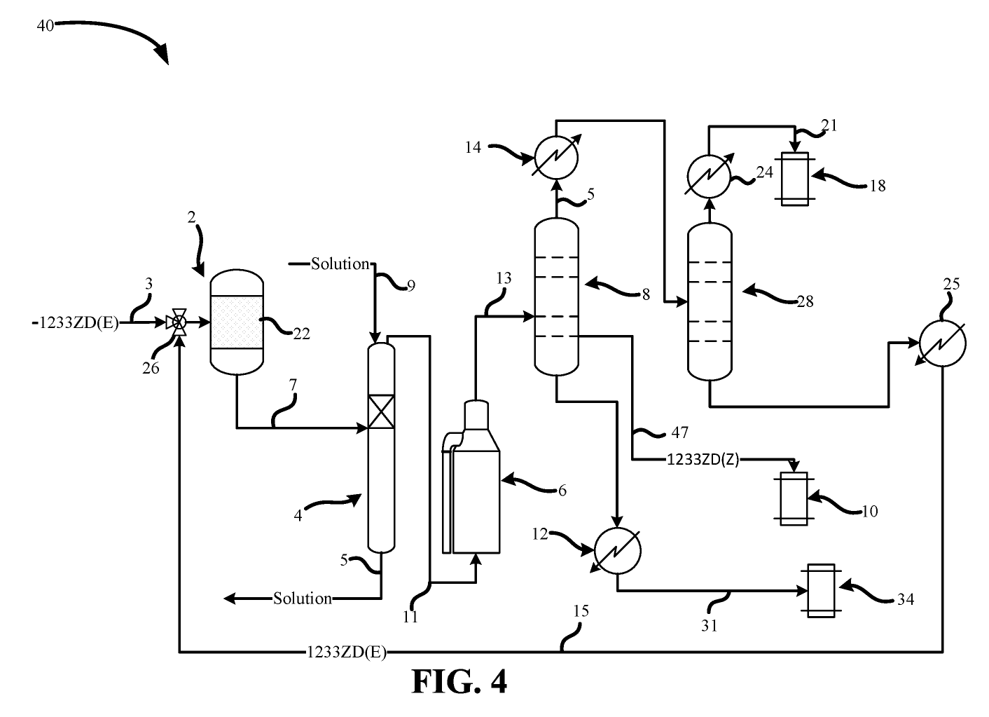

FIG. 4 also illustrates a process flow diagram similar to that of FIG. 2, but where process flow 40 has the stream that contains a majority of 1233zd(Z) being removed from the stripping section of distillation column 8 as stripping section 1233zd(Z) stream 47 and then collected in 1233zd(Z) container 10. High boiling co-products stream 33 is removed as the bottoms of distillation column 8 and is collected in high boiling co-products container 34 and may be used in other processes, sold, and/or disposed of.

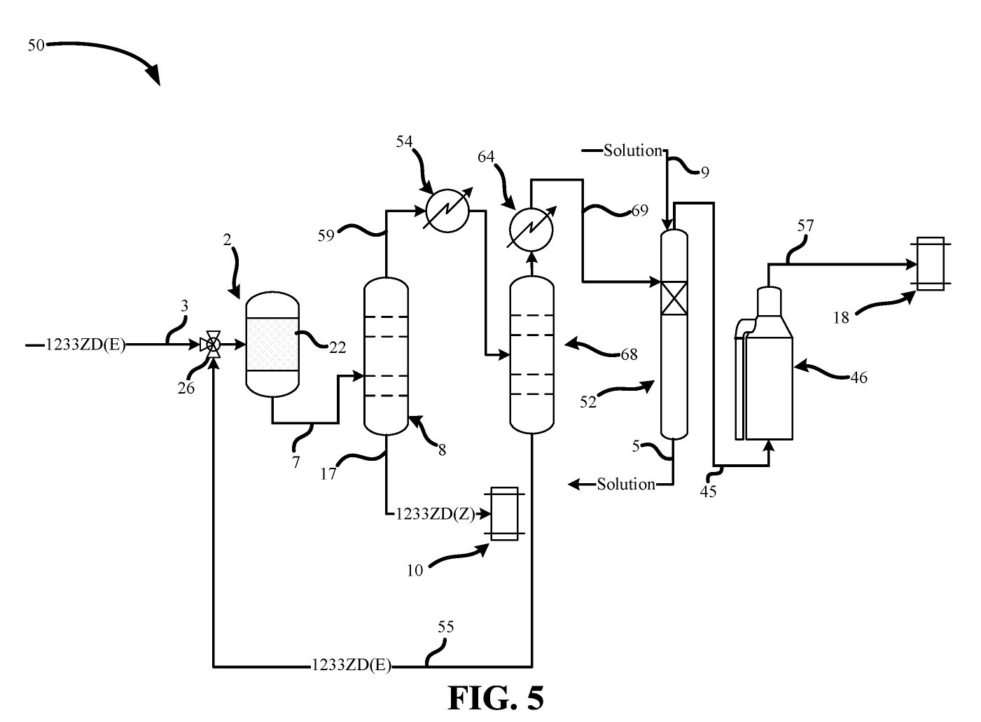

In various embodiments, prior to sending the reacted fluid to scrubber 44, multiple separation processes may be used in series (i.e., two or more) to remove greater quantities of 1233zd(Z) and, thus, reduce the amount of 1233zd(Z) that may contact the solution 9. For example, FIGS. 5-10 are various process flow diagrams depicting various processing methods where distillation occurs before scrubbing a product stream with solution stream 9.

In some embodiments, it may be preferable to distill the reactor fluid stream 7 prior to scrubbing with a water or a weak caustic solution to prevent the breakdown of the less stable 1233zd(Z) and/or prevent the formation of HCl and HF during the separation process.

For example, FIG. 5 is a process flow diagram showing process 50 of manufacturing and separating (Z)-1-chloro-3,3,3-trifluoropropene that includes at least a second column to separate the overhead of the first distillation column series before the separated product stream is scrubbed. After reacted fluid stream 7 is removed from isomerization reactor 2, reacted fluid stream 7 may be distilled in distillation column 8. (Z)-1-chloro-3,3,3-trifluoropropene may be removed as bottoms 1233zd(Z) stream 17, while overhead stream 59 may be sent through cooler or reboiler 54 to second distillation column 68. In second distillation column 68, the (E)-1-chloro-3,3,3-trifluoropropene may be separated from other low boiling co-products and recycled as bottoms 1233zd(E) recycle stream 55. Overhead stream 59 may be then sent to scrubber 44 where overhead 69 may be scrubbed with solution stream 59 in scrubber 52, which may remove various contaminants, such as HF and/or HCl in scrubber product stream 5. The scrubbed overhead stream 45 may then be dried in drier 46 (e.g., with a desiccant) and then pumped as dried stream 67 into low boiling co-product collection container 18.

For example, in some embodiments, using a series of distillation columns may result in a bottoms 43 having a greater than 90 wt. % purity of (Z)-1-chloro-3,3,3-trifluoropropene, a greater than 95 wt. % purity of (Z)-1-chloro-3,3,3-trifluoropropene, a greater than 98 wt. % purity of (Z)-1-chloro-3,3,3-trifluoropropene, or an essentially pure (Z)-1-chloro-3,3,3-trifluoropropene product in container 10.

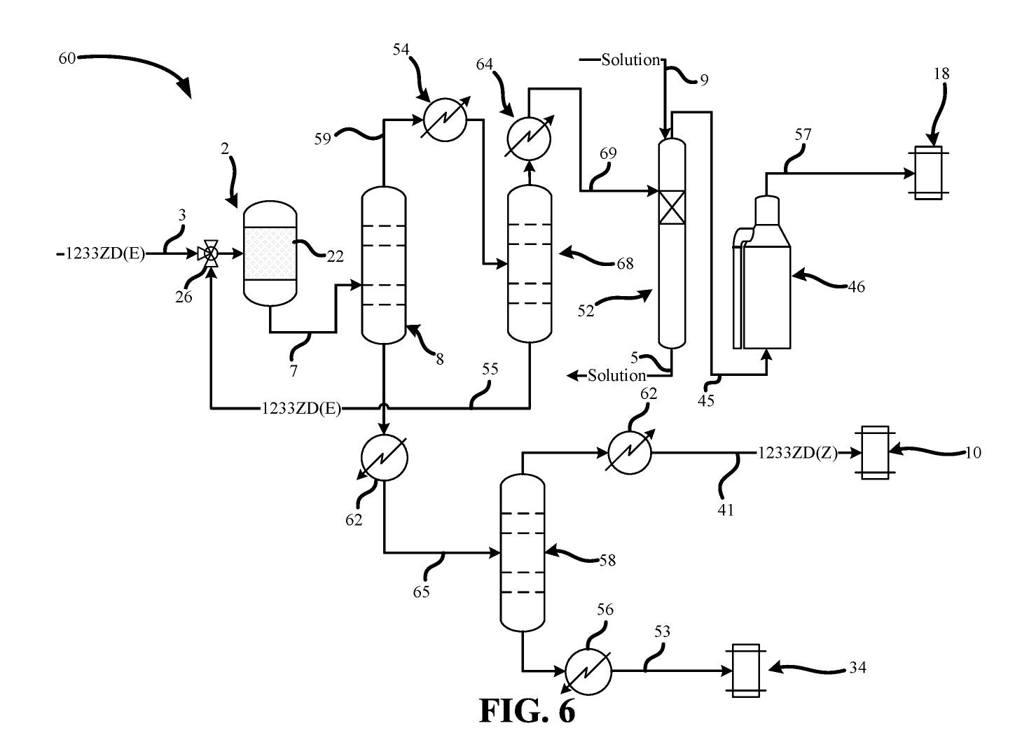

In some embodiments, such as process flow 60 illustrated in FIG. 6, subsequent separation may occur for both the overhead 59 of distillation column 8 and bottoms 65. Bottoms 65 may be heated or cooled by heat exchanger 62 and sent into distillation column 58. The overhead 1233zd(Z) stream 41 may be heated or cooled in heat exchanger 62 and sent to 1233zd(Z) collection container 10. The bottoms high boiling co-products stream 53 may be heated or cooled by heat exchanger 56 and then collected in high boiling co-products container 34.

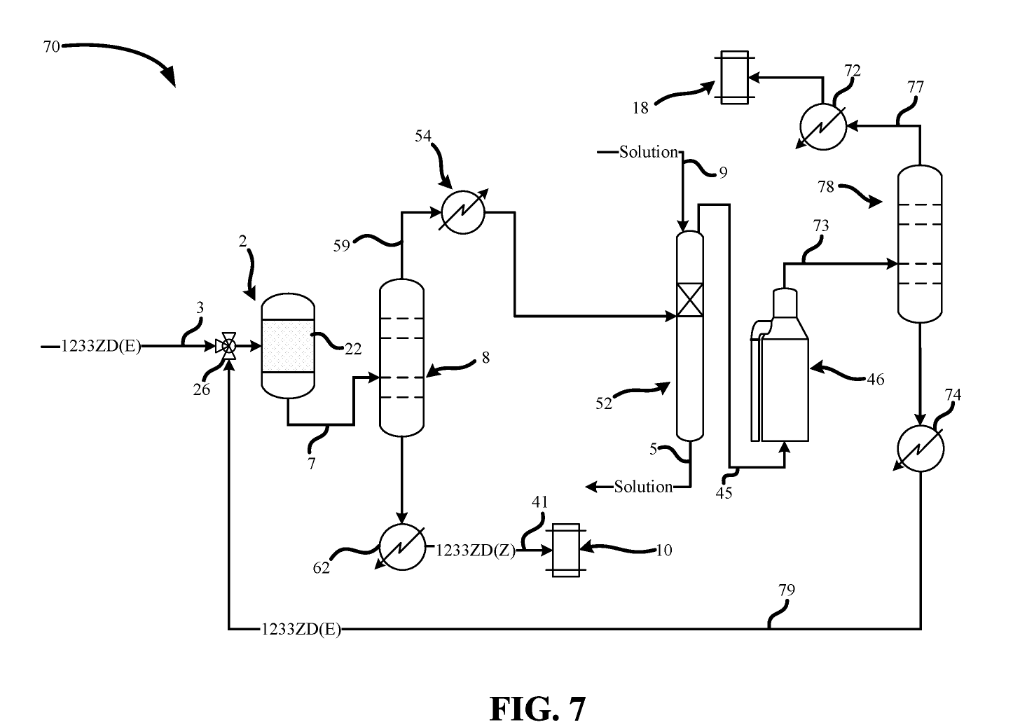

FIG. 7 illustrates process flow 70, which is similar to the process flow depicted in FIG. 5, but the scrubbed product stream 45 that is sent to dryer 46 may then be sent to distillation column 78 as dried product stream 73. In distillation column 78, the dried product stream 73 may be separated into high boiling co-products overhead stream 77 and bottoms 1233zd(E) recycle stream 79, which may be reheated or cooled in heat exchanger 74 and recycled back with reactant stream 3 via mixer valve 26. High boiling co-products overhead stream 77 may be heated or cooled with heat exchanger 72 and then collected in high boiling co-products container 34.

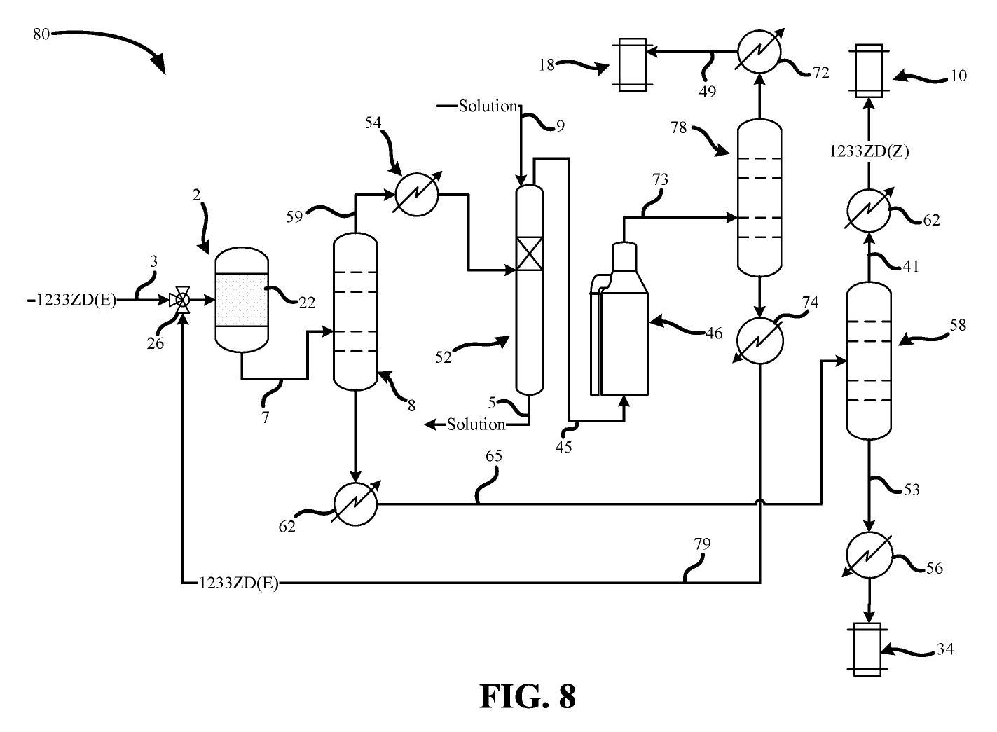

FIG. 8 illustrates process flow 70, which is a combination of process flow 60 and 70 (depicted in FIGS. 6 and 7 respectively) where the overhead stream 59 is processed similarly to the process flow 70 (shown in FIG. 7) and the bottoms 65 is processed similarly to that shown in process flow 60 (shown in FIG. 6).

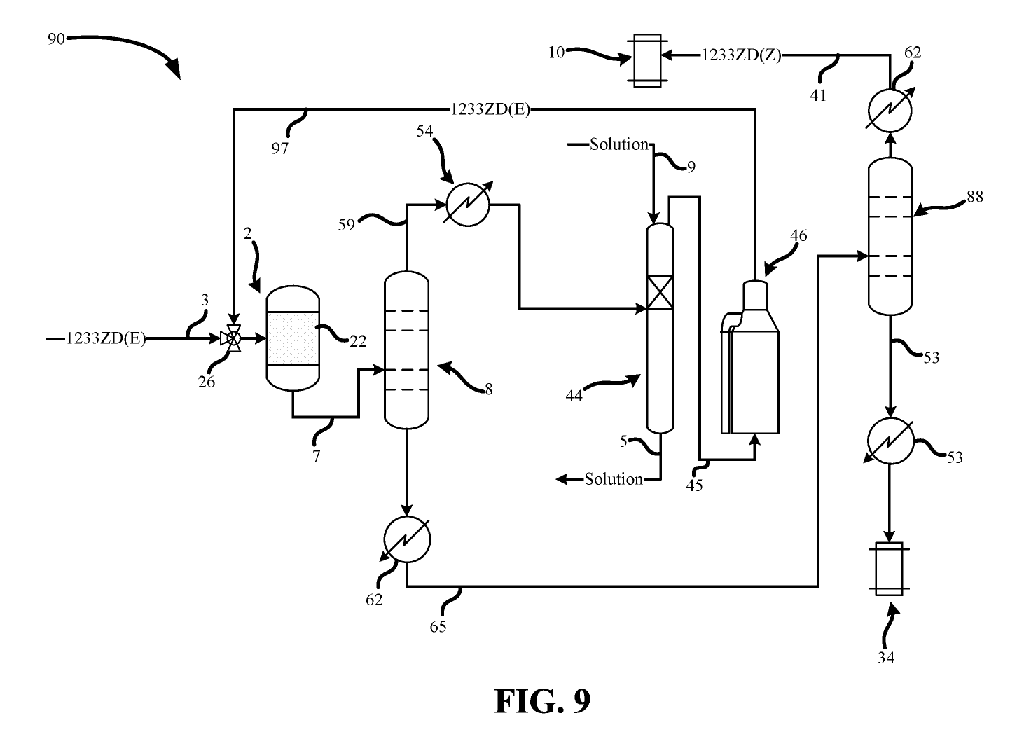

FIG. 9 illustrates process flow 90 that is similar to process flow 80 shown in FIG. 8, but the scrubbed overhead stream 45 that is sent to dryer 46 is subsequently recycled directly back to be combined with reactant stream 3 via mixer valve 26 as dried 1233zd(E) recycle stream 97.

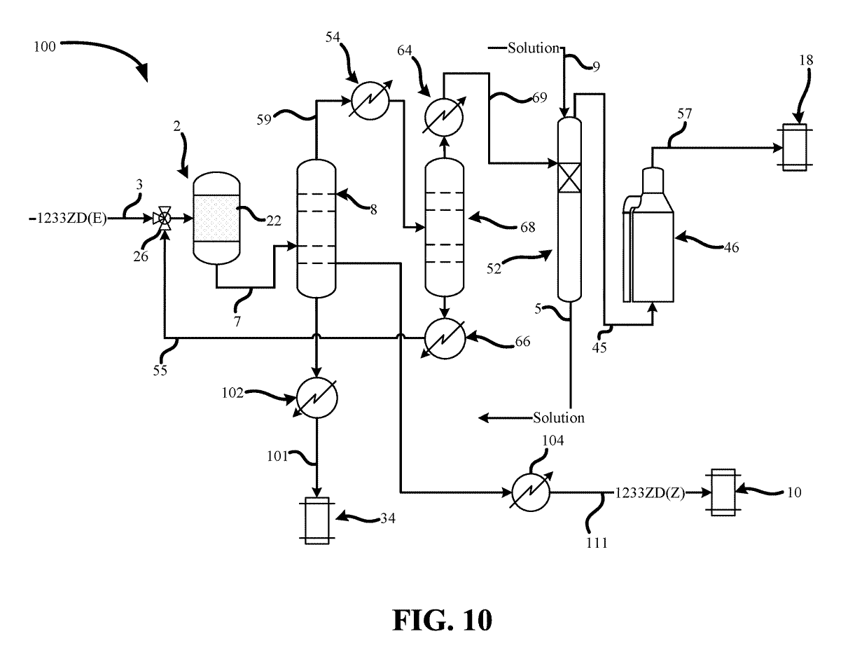

FIG. 10 illustrates yet another process flow 100, which is similar to process flow 60 depicted in FIG. 6, except distillation column 8 has both a stripping section 1233zd(Z) stream 111 and a bottoms high boiling point co-products stream 101. As depicted in FIG. 10, stripping section 1233zd(Z) stream 111 may be heated or cooled by heat exchanger 104 and collected in 1233zd(Z) container 10, while high boiling point co-products stream 101 may be heated or cooled by heat exchanger 102 and collected in high boiling point co-products container 34.

As used herein the term “separation” recovery processes may utilize various separation techniques (e.g., decanting, liquid-liquid separation, distillation, and flash distillation) and may also utilize the unique properties of azeotropic or azeotrope-like compositions. Moreover, as used herein, flash distillation may be understood to include pre-flashing, which may be used to reduce the load on flash distillation separator. Separating various phases (e.g., the HF phase and the organic phase) may include at least one of decanting, centrifuging, liquid-liquid extraction, vapor-liquid-liquid extraction (VLLE), distilling, flash distilling, or combinations thereof.

Also, the term “scrubber” as used herein, may be understood to include control devices or materials that inject a dry reagent or slurry into a dirty exhaust stream to “wash out” acid gases, such as solution 9. The solution 9 is not particularly limited and may include water or any suitable caustic solution with a pH below or equal to about 10, such as NaOH, KOH, or Ca(OH)₂. The dryer is also not particularly limited and may include desiccants such as molecular sieves, calcium trifluoropropyne, calcium sulfate, sulfuric acid, or combinations thereof.

As used herein, the phrase “within any range defined between any two of the foregoing values” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

The Examples below along with their accompanying data can help to illustrate various advantages of some embodiments disclosed herein.

### EXAMPLES 1-3

In Examples 1-3, the selectivity and conversion of 1233zd(E) to 1233zd(Z) was measured and reported for the catalysts 316 stainless steel, Iconel 625, and Monel 400.

### EXAMPLE 1

### 316 Stainless Steel Propak Distillation Packing

| Temp. | selectivity to | Conversion of |
|---|---|---|
| (° C.) | 1233zd(Z) (%) | 1233zd(E) (%) |
| 400 | 97.76% | 11.21% |
| 450 | 94.70% | 16.60% |

### EXAMPLE 2

### Inconel 625 Mesh

| Temp. | selectivity to | Conversion of |
|---|---|---|
| (° C.) | 1233zd(Z) (%) | 1233zd(E) (%) |
| 400 | 96.86% | 3.52% |
| 450 | 97.15% | 11.57% |

### EXAMPLE 3

### Monet 400 Propak Distillation Packing

| Temp. | selectivity to | Conversion of |
|---|---|---|
| (° C.) | 1233zd(Z) (%) | 1233zd(E) (%) |
| 400 | 97.27% | 4.64% |
| 450 | 96.63% | 11.59% |

As can be seen above, of the zero-valent alloys tested, stainless steel and Inconel 625 catalyst demonstrate highest selectivity.

In Example 4, the results of which are shown below in Table 1, the evaluated performance of 316 Stainless Steel catalyst was evaluated. The isomerization reactor has an internal diameter of 4″ and a length of 31″. The catalyst used was 0.25″ Stainless Steel Propak (7 L).

TABLE 1
Evaluated Performance of 316 Stainless Steel Catalyst

| | | | | | | Select. to |
|---|---|---|---|---|---|---|
| | | 1233zd(E) | Conv. of | Select. to | Select. to | Dimers |
| Temp. | Press. | feed rate | 1233zd(E) | 1233zd(Z) | 244fa | (C₆H₃F₆Cl) |
| (° C.) | (psig) | (lb./hr) | (Mole %) | (Mole %) | (Mole %) | (Mole %) |
| 350 | 2 | 1.0 | 13.11 | 97.77 | 0.13 | 1.39 |
| 350 | 2 | 1.5 | 12.88 | 97.96 | 0.13 | 1.49 |
| 375 | 2 | 2.0 | 14.42 | 95.47 | 0.10 | 3.38 |
| 400 | 2 | 2.0 | 15.95 | 90.32 | 0.05 | 7.84 |
| 350 | 30 | 1.0 | 13.95 | 91.89 | 0.07 | 6.57 |
| 350 | 30 | 1.5 | 13.35 | 96.35 | 0.07 | 2.50 |
| 350 | 30 | 2.0 | 13.22 | 96.04 | 0.08 | 2.63 |
| 375 | 30 | 2.0 | 13.90 | 93.62 | 0.04 | 4.41 |
| 380 | 30 | 2.0 | 14.31 | 91.27 | 0.03 | 6.38 |
| 390 | 30 | 2.0 | 15.33 | 90.45 | 0.03 | 6.69 |
| 400 | 30 | 2.0 | 16.79 | 80.43 | 0.02 | 15.44 |
| 350 | 30 | 2.0 | 13.22 | 96.04 | 0.08 | 2.63 |
| 350 | 30 | 2.6 | 10.12 | 97.48 | 0.04 | 0.42 |
| 360 | 30 | 2.6 | 12.32 | 97.57 | 0.04 | 0.91 |

### EXAMPLE 5

Similarly, in Example 5, the fluorinated amorphous Cr₂O₃ catalyst was evaluated and the average selectivity and average conversion is shown in Table 2 below.

TABLE 2
Average Conversion (%) and Average Selectivity (%) of Fluorinated Amorphous Cr₂O₃

| Temperature | Av. Conv % | Av. Selectivity % | | | | | | |
|---|---|---|---|---|---|---|---|---|
| C. | t-1233zd | t-1234ze | 245fa | c-1234ze | c-1233zd | 1232 | 243fa | others |
| 150 | 5.57 | 1.50 | 0.38 | 0.08 | 97.20 | 0.47 | 0.00 | 0.36 |
| 200 | 7.98 | 5.35 | 0.10 | 0.65 | 91.83 | 1.64 | 0.01 | 0.43 |
| 225 | 9.17 | 7.00 | 0.55 | 0.99 | 88.82 | 1.73 | 0.10 | 0.80 |
| 250 | 9.71 | 7.72 | 0.16 | 1.18 | 87.59 | 2.10 | 0.14 | 1.10 |
| 300 | 12.11 | 11.40 | 0.47 | 2.25 | 81.14 | 1.83 | 0.14 | 2.77 |

Feed composition: 100% t-1233zd; pressure 50 psig; fluorinated Cr2O3; amount loaded 410 g; flow rate 0.6 lb./hr; reactor dimensions: 1″ ID × 31″ L. Each temperature was run for at least 20 hours.

### EXAMPLES 6-9

In Examples 6-9, low pressure batch distillation data was taken to analyze the selectivity of various catalysts.

In Example 6, the catalyst tested was unfluorinated amorphous Cr₂O₃. The batch was run at a temperature of 250° C. The selectivity was found to be below 92%, but the major co-products were found to be recoverable and were also found to be reusable as intermediates or products themselves (e.g., 1234ze(E), 245fa, 1234ze(Z), 1232zd).

In Example 7, the catalyst tested was fluorinated crystalline Cr₂O₃. The batch was run at a temperature of 250° C. The selectivity was below 80%, but the major co-products were found to be recoverable and were also found to be usable as intermediates or products themselves (e.g., 234ze(E), 245fa, 1234ze(Z), 1232zd).

In Example 8, the catalyst tested was Alumina or Aluminum Fluoride. The batch was run at a temperature of 250° C. The selectivity was found to be below 80%, but major co-products were found to be recoverable and usable as intermediates or products themselves (i.e. 1234ze(E), 245fa, 1234ze(Z), 1232zd).

In Example 9, the catalyst tested was FeC13/Carbon. The batch was run at a temperature of 250° C. The selectivity was observed to be about 80%, but not stable meaning that the catalyst deactivated quickly as seen by a steady decrease in 1233zd(E) conversion and 1233zd(Z) selectivity.

### EXAMPLE 10

In Example 10, the scrubbing of the reactor effluent was examined. The reactor effluent material composed primarily of 88% 1233zd(E) and 12% 1233zd(Z) with some residual byproducts at low levels was passed through a packed scrubber column through which a warm, weak KOH solution was being circulated to remove residual HF by-product. The pH of the KOH solution was controlled to remain between a pH of 8 and 10 to minimize decomposition of 1233zd(Z) to form trifluoropropyne. The stream was then passed through a 3 A molecular sieve drying system to remove any moisture that was picked up when it passed through the KOH solution.

After drying, the acid free 1233zd crude was collected in a tank awaiting separation of the 2ea×1233zd isomers by distillation.

### EXAMPLE 11

In Example 11, a recycle column of 1233zd(E) was examined to determine whether recycled unreacted 1233zd(E) could be reused and, thus, improve the yield of various embodiments. A distillation column was used to separate the scrubbed 1233zd(Z) product (having a boiling point of about 39° C.) from the 1233zd(E) (having a boiling point of about 19° C.) so that the product could be collected for final distillation while the unreacted 1233zd(E) was recycled back to the reaction.

The distillation was performed in a 6″ diameter by twenty-one foot tall packed column with ¼″ pro-pak dump packing. A 10-gallon thermosyphon steam reboiler and a shell-and-tube condenser with cooling water was used for heat transfer. At 60 pounds per hour of continuous feed, the system recovered 1233zd(Z) with a purity of 98.5% in the bottom stream, and recycled 1233zd(E) overhead with 99% product quality. The 1233zd(Z) was then stored for final distillation to pure product.

Given that the purity of the recycled 1233zd(E) off the overhead, methods using recycled unreacted 1233zd(E) may be used to improve yield of the manufacturing of 1233zd(Z) according to various embodiments.

### EXAMPLE 13

Example 13 was performed to explore distillation of the 1233zd(Z) product using distillation runs using a pilot plant distillation column. The column used a heat exchanger with regulated 40 psig steam as a reboiler, and a 500-gallon flash tank that was initially charged with the crude mixture of components. Column itself was 12″ diameter by thirty-five feet tall column, packed with GOODLOE® structured packing that resulted in approximately 75 stages of separation. The column could be run up to 200 psig, but because of the high boiling point of the 1233zd(Z) material, it operated at a relatively low pressure of 15 psig to minimize the temperature required to run. The steam fed to the reboiler was at 40 psi and approximately 200 lb./hr. The overhead cooling was controlled in cascade with the column pressure at 15 psig. In doing this, the reflux was maintained between 2200 and 2500 lb./hr.

Because material was already sent through a recycle column, the 1233zd(Z) composition in this example was 98% 1233zd(Z), with approximate 1% 1233zd(E) and 100 PPM of trifluoropropyne as lights components. The remaining 1% was made up of a number of heavier components including 1232 isomers and dimers formed in the reaction. Charge quantity in this example to system was 4,000 lb. per batch.

In some runs, a lights (vapor) cut was first taken VERY slowly at less than 10 lb/hr to try and remove the 1233zd(E) as a recycle stream to be returned to the intermediate hold tanks. A cut of 200 lb. was removed to take out the lower boiling impurities.

The main cut was collected at about 200 lb/hr to collect the purified 1233zd(Z) material. Once the 1233zd(E) and other light materials were removed, the main product cut was an easy separation from any of the heavy components. No other impurity was found to be more than 10 PPM in the product cuts.

The target impurity levels were as follows:

| | |
|---|---|
| 1233zd(E) | no more than 1000 ppm |
| 245fa (1,1,1,3,3-pentafluoropropane) | no more than 500 ppm |
| 244fa (3-Chloro-1,1,1,3-tetrafluoropropane) | no more than 50 ppm |
| 243fa (3,3-dichloro-1,1,1-trofluoropropane) | no more than 50 ppm |
| 3,3,3-Trifluoropropyne | no more than 15 ppm |
| Trans-1234ze | no more than 100 ppm |
| (Trans-1,3,3,3-tetrafluoropropene) | |
| Cis-1234ze (Cis-1,3,3,3-tetrafluoropropene) | no more than 50 ppm |
| 1232 isomers (dichlorodifluoropropene) | no more than 50 ppm |
| 1234zc (1,1,3,3-tetrafluoropropene) | no more than 50 ppm |
| 1224 isomers | no more than 20 ppm |
| (1-chloro-2,3,3,3-tetrafluoropropene) | |
| 1233zd dimers (C₆H₃ClF₆) | no more than 20 ppm |
| 1233xf (2-chloro-3,3,3-trifluoropropene) | present in trace amounts |
| | no more than 50 ppm |
| 244bb (2-chloro-1,1,1,2-trifluoropropene) | present in trace amounts |
| | no more than 50 ppm |

Distillation was halted when there was not enough material in the system to provide motive force for reflux. Product recovery on a single batch basis was about 75%. Further batch distillation can be run charging more material into the system with residual undistilled material still remaining to increase overall recovery.

Thus, as can be seen from above, the manufacture and separation of (Z)-1-chloro-3,3,3-trifluoropropene (HCFO-1233zd(Z)) from (E)-1-chloro-3,3,3-trifluoropropene (HCFO-1233zd(E)) with the use of either a low temperature or high-temperature catalyst may be accomplished. Furthermore, the cost-efficient separation processes disclosed herein may allow for the recycling of unused reagents to improve overall yields of (Z)-1-chloro-3,3,3-trifluoropropene.

While this disclosure has been described as having various exemplary designs, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. § 112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A method of manufacturing (Z)-1-chloro-3,3,3-trifluoropropene comprising:
contacting a reactant fluid stream containing (E)-1-chloro-3,3,3-trifluoropropene with a catalyst to form a reacted fluid stream comprising (Z)-1-chloro-3,3,3-trifluoropropene;
contacting the reacted fluid stream with a scrubbing fluid to remove a byproduct from the reacted fluid stream;
removing the scrubbing fluid from the reacted fluid stream;
separating (Z)-1-chloro-3,3,3-trifluoropropene from unreacted (E)-1-chloro-3,3,3-trifluoropropene in the reacted fluid stream; and
recycling the unreacted (E)-1-chloro-3,3,3-trifluoropropene with the reactant fluid stream containing (E)-1-chloro-3,3,3-trifluoropropene.

2. The method of claim 1, wherein the separating (Z)-1-chloro-3,3,3-trifluoropropene from unreacted (E)-1-chloro-3,3,3-trifluoropropene in the reacted fluid stream is done by distillation.

3. The method of claim 2, wherein the distillation is done with a plurality of distillation columns.

4. The methods of claim 3, wherein the distillation columns are in series.

5. The method of claim 1, wherein the scrubbing fluid comprises water, a low-caustic composition, or a mixture thereof.

6. The method of claim 1, wherein the dryer comprises a desiccant.

7. The method of claim 1, wherein the dryer comprises molecular sieves, a mesoporous material, a macroporous material, inorganic anhydrites, or a combination thereof.

8. The method of claim 1, wherein the catalyst is a high-temperature catalyst or a low temperature catalyst.

9. The method of claim 2, wherein the catalyst is a low temperature catalyst and the low temperature catalyst is a chromium based low temperature catalyst, an aluminum based low temperature catalyst, a halogenated metal, a metal oxide, or a combination thereof.

10. The method of claim 9, wherein the low temperature catalyst is a chromium based low temperature catalyst and the chromium based low temperature catalysts is chromium oxide, chromium oxyfluoride, chromium fluoride, or a combination thereof.

11. The method of claim 1, wherein the contacting the fluid stream containing (E)-1-chloro-3,3,3-trifluoropropene with the catalyst to form the reacted fluid stream comprising (Z)-1-chloro-3,3,3-trifluoropropene is done at a temperature below about 600° C.

12. The method of claim 1, wherein the contacting the fluid stream containing (E)-1-chloro-3,3,3-trifluoropropene with the catalyst to form the reacted fluid stream comprising (Z)-1-chloro-3,3,3-trifluoropropene is done at an operating pressure between about 4 psia to about 214 psia.

13. A method of manufacturing (Z)-1-chloro-3,3,3-trifluoropropene comprising:
contacting a reactant stream containing (E)-1-chloro-3,3,3-trifluoropropene with a catalyst to form a reacted fluid stream containing (Z)-1-chloro-3,3,3-trifluoropropene;
separating (Z)-1-chloro-3,3,3-trifluoropropene from unreacted (E)-1-chloro-3,3,3-trifluoropropene in the reacted fluid stream to form a separated reacted fluid stream containing unreacted (E)-1-chloro-3,3,3-trifluoropropene;
contacting the separated reacted fluid stream containing unreacted (E)-1-chloro-3,3,3-trifluoropropene with a scrubbing fluid to remove one or more byproducts from the separated reacted fluid stream;
removing the scrubbing fluid from the separated reacted fluid stream containing unreacted (E)-1-chloro-3,3,3-trifluoropropene; and
recycling the unreacted (E)-1-chloro-3,3,3-trifluoropropene with the reactant fluid stream containing (E)-1-chloro-3,3,3-trifluoropropene.

14. The method of claim 13, further comprising separating hydrogen halides from the separated reacted fluid stream containing unreacted (E)-1-chloro-3,3,3-trifluoropropene.

15. The method of claim 14, wherein the hydrogen halides are separated from the separated reacted fluid stream containing unreacted (E)-1-chloro-3,3,3-trifluoropropene after the separated reacted fluid stream containing unreacted (E)-1-chloro-3,3,3-trifluoropropene is contacted with the scrubbing fluid.

16. The method of claim 13, wherein the separating (Z)-1-chloro-3,3,3-trifluoropropene from unreacted (E)-1-chloro-3,3,3-trifluoropropene in the reacted fluid stream is done by distillation.

17. The method of claim 16, wherein the distillation is done with a plurality of distillation columns.

18. The methods of claim 17, wherein the distillation columns are in series.

19. The method of claim 13, wherein the scrubbing fluid comprises water, a low-caustic composition, or a mixture thereof.

20. The method of claim 13, wherein the dryer molecular sieves, a mesoporous material, a macroporous material, inorganic anhydrites, or a combination thereof.
